(19) United States
(12) Patent Application Publication
(10) Pub. No.: US 2009/0023643 A1
(43) Pub. Date: Jan. 22, 2009

(54) Methods For Treating Acute Myocardial Infarction By Administering Calcitonin Gene Related Peptide And Compositions Containing The Same

(75) Inventors: Jeffrey L. Southard, Lenexa, KS (US); George Lee Southard, Sanibel, FL (US)

Correspondence Address: MORRISON & FOERSTER LLP, 755 PAGE MILL RD, PALO ALTO, CA, 94304-1018, US

(73) Assignee: VasoGenix Pharmaceuticals, Inc., Lenexa, TX (US)

(21) Appl. No.: 10/586,036

(22) PCT Filed: Jan. 13, 2005
(86) PCT No.: PCT/US2005/001230

§ 371 Date: Aug. 19, 2008

Related U.S. Application Data
(60) Provisional application No. 60/560,745, filed on Jan. 13, 2004.
(60) Provisional application No. 60/608,945, filed on Jan. 13, 2004.
(60) Provisional application No. 60/565,056, filed on Apr. 23, 2004.

(51) Int. Cl. A61K 38/17; A61P 9/00

(52) U.S. Cl. 514/12

(57) ABSTRACT

This invention relates to methods of treating acute myocardial infarction by administering calcitonin gene related peptide (CGRP). This invention also relates to preventing an acute myocardial infarction by administering calcitonin gene related peptide (CGRP). This invention further relates to compositions of CGRP for use in such methods. This invention also relates to the use of calcitonin gene related peptide in the manufacture of medicament for treating or preventing an acute myocardial infarction in a subject or for treating a subject suspected of having an acute myocardial infarction.

### CROSS-REFERENCE TO RELATED APPLICATIONS

This application claims the benefit of U.S. Provisional Application Ser. No. 60/565,056, filed Apr. 23, 2004, U.S. Provisional Application Ser. No. 60/560,745, filed Jan. 13, 2004 and U.S. Provisional Application Ser. No. 60/608,945, filed Jan. 13, 2004, the disclosures of which are all incorporated herein by reference in their entirety.

### STATEMENT REGARDING FEDERALLY SPONSORED RESEARCH OR DEVELOPMENT

Not applicable.

### FIELD OF THE INVENTION

This invention relates to methods and compositions for treating cardiovascular disease, in particular this invention relates to methods and compositions for treating an acute myocardial infarction by administering calcitonin gene related peptide. This invention also relates to the use of calcitonin gene related peptide in the manufacture of medicament for treating or preventing an acute myocardial infarction in a subject or for treating a subject suspected of having an acute myocardial infarction.

### BACKGROUND OF THE INVENTION

Despite improvements in the diagnosis and management of acute myocardial infarction (AMI) in the past few decades it continues to be a major health concern. For example, each year in the United States nearly one million people have a AMI and an even greater number are admitted for consideration of the diagnosis (Braunwald, E et al (ed.) In Heart Disease—A Textbook of Cardiovascular Medicine, 6^{th} Edition, (2001) W.B. Saunders Company, Philadelphia; Chapter 35; Crawford, M H (ed.), In Current Diagnosis and Treatment of Cardiology, 2^{nd} Edition, (2003) Lange Medical Books/McGraw Hill, New York; Chapter 5). Of these occurrences of acute myocardial infraction, approximately one third results in the death of the patient. (See Brunwald, E et al).

AMI is a clinical condition associated with the death of myocardial tissue generally resulting from a prolonged imbalance between oxygen supply and demand. The clinical indicia for AMI includes a combination of symptoms (e.g., chest pain), characteristic electrocardiographic changes and an increase in plasma levels of intracellular enzymes released by the myocytes as they become necrotic. Generally, an AMI results from an occlusion of the coronary vessels (e.g., thrombosis from plaque rupture) but may be caused by a variety of other factors as well (e.g., vascular injury, infective endocarditis, cocaine abuse). Total occlusion of coronary vessels for more than about 3-6 hours can result in irreversible tissue damage but reperfusion within this period can salvage the tissue and reduce morbidity and mortality. Paradoxically, the restoration of coronary blood flow to the area of infarction with reperfusion therapies can result in further tissue damage known as reperfusion injury.

Current pharmacological therapies recommended jointly by the American Heart Association (AHA) and the American College of Cardiology (ACC) for treating AMI include thrombolytic agents (e.g., streptokinase, TNK-tissue plasminogen activator) to break up blood clots, nitrates to dilate the vasculature thus reducing the heart's work load and need for oxygen, and antiplatelet agents (e.g., aspirin, clopidogrel, glycoprotein IIb/IIIa inhibitors) to inhibit platelet aggregation. Other recommended therapies include beta-blockers for arrhythmia, ACE inhibitors to lower blood pressure and reduce the heart's workload, and calcium channel blockers.

Other interventions for acute myocardial infarction include percutaneous transluminal coronary angioplasty (PTCA) and coronary artery bypass graft (CABG) surgery. Although these procedures can restore blood flow to affected myocardial tissue, they transiently reduce blood flow through the target artery. Clinical risk factors, including age, diabetes, preexisting cardiovascular diseases, and renal insufficiency significantly increase the potential of serious ischemic events during and immediately following both coronary angioplasty and CABG procedures. (Smith S C Jr. et al, (2001). ACC/AHA Guidelines for Percutaneous Coronary Intervention: a Report of the American College of Cardiology/American Heart Association Task Force on Practice Guidelines (Committee to Revise the 1993 Guidelines for Percutaneous Transluminal Coronary Angioplasty). Journal of The American College of Cardiology 37:2239i-lxvi). It is estimated that up to 20% of high risk patients undergoing these procedures will experience some form of post-procedural ischemic complication, such as vasospasms, no-reflow, or renal insufficiency, that can cause tissue damage and have the potential to cause AMIs, renal failure or death. [Ryan T J, et al (1999). ACC/AHA Guidelines for the Management of Patients with Acute Myocardial Infarction: 1999 Update: a Report of the American College of Cardiology/American Heart Association Task Force on Practice Guidelines (Committee on Management of Acute Myocardial Infarction) Journal of the American College of Cardiology 33:1756-824; Smith S C Jr. et al, (2001). ACC/AHA Guidelines for Percutaneous Corona Intervention: a Report of the American College of Cardiology/American Heart Association Task Force on Practice Guidelines (Committee to Revise the 1993 Guidelines for Percutaneous Transluminal Coronary Angioplasty)].

These current therapies, therefore, have a side effect that plays a major role in myocardial tissue survival, namely, ischemic-reperfusion injury. Ischemic-reperfusion injury results in myocardial cell death, the extent of which is directly proportional to the duration of the ischemic insult.

Accordingly, there is a need for additional therapeutic treatments to supplement and/or complement existing therapies.

### SUMMARY OF THE INVENTION

This invention generally relates to a method of treating an acute myocardial infarction in a subject, or treating a subject suspected of having a myocardial infarction or preventing an acute myocardial infarction in a subject, comprising administering CGRP to a subject in need of such treatment and compositions for use in such methods.

In one embodiment, the invention relates to a method of treating an acute myocardial infarction in a subject, comprising administering CGRP to a subject in need of such treatment CGRP at a rate between about 0.8 ng/kg/min to about 16 ng/kg/min for up to about 24 hours.

In yet another embodiment, the invention relates to a method of treating an acute myocardial infarction in a subject, comprising administering CGRP to a subject in need of such treatment at a rate between about 4 ng/kg/min to about 10 ng/kg/min, such as for example, about 8 ng/kg/min for up to about 24 hours.

In another embodiment, the invention relates to a method of treating an acute myocardial infarction in a subject, comprising administering CGRP to a subject in need of such treatment at a rate sufficient to achieve steady state plasma levels at between about 16 pg/ml to between about 314 pg/ml, for up to about 24 hours or between about 79 pg/ml to about 196 pg/ml, such as for example about 157 pg/ml, for up to about 24 hours.

In yet another embodiment the method relates to treating a non-ST elevated acute myocardial infarction by administering CGRP to a subject in need of such treatment.

In another embodiment the method relates to treating an ST elevated acute myocardial infarction by administering CGRP to a subject in need of such treatment.

In another embodiment, the invention relates to a method of treating a subject suspected of having an acute myocardial infarction, comprising administering CGRP to a subject in need of such treatment at a rate between about 0.8 ng/kg/min to about 16 ng/kg/min for up to about 24 hours.

In another embodiment, the invention relates to a method of treating a subject suspected of having an acute myocardial infarction, comprising administering CGRP to a subject in need of such treatment CGRP at a rate between about at a rate between about 4 ng/kg/min to about 10 ng/kg/min, such as for example, about 0.8 ng/kg/min for up to about 24 hours.

In another embodiment, the invention relates to a method of treating a subject suspected of having an acute myocardial infarction, comprising administering CGRP to a subject in need of such treatment at a rate sufficient to achieve steady state plasma levels at between about 16 pg/ml to between about 314 pg/ml, for up to about 24 hours or between about 79 pg/ml to about 196 pg/ml, such as for example about 157 pg/ml, for up to about 24 hours.

In another embodiment, the invention relates to a method of preventing an acute myocardial infarction in a subject in need of such treatment, comprising administering CGRP to a subject in need of such treatment at a rate between about 0.8 ng/kg/min to about 10 ng/kg/min continuously as needed.

In another embodiment, the invention relates to a method of preventing an acute myocardial infarction in a subject in need of such treatment, comprising administering CGRP to a subject in need of such treatment at a rate sufficient to achieve steady state plasma levels at between about 16 pg/ml to about 196 pg/ml continuously as needed.

In another embodiment, the invention provides compositions, such as for example, intravenous formulations and controlled release formulations, and kits comprising CGRP for use in any of the methods of the invention.

This invention also relates to the use of calcitonin gene related peptide in the manufacture of medicament for treating or preventing an acute myocardial infarction in a subject or for treating a subject suspected of having a myocardial infarction.

The invention also provides any of the compositions and kits described for any use described herein whether in the context of use as medicament and/or use for manufacture of a medicament.

All references cited herein, including patent applications and publications, are incorporated by reference in their entirety.

### DETAILED DESCRIPTION OF THE INVENTION

The practice of the present invention will employ, unless otherwise indicated, conventional techniques of clinical medicine, pharmacology and molecular biology (including recombinant techniques), which are within the skill of the art. Such techniques are explained fully in the literature, such as, for example, Braunwald, E et al (ed.) In Heart Disease—A Textbook of Cardiovascular Medicine, 6^{th} Edition, (2001) W.B. Saunders Company, Philadelphia; Chapter 35; Crawford, M H (ed.), In Current Diagnosis and Treatment of Cardiology, 2^{nd} Edition, (2003) Lange Medical Books/McGraw Hill, New York; Chapter 5; Molecular Cloning: A Laboratory Manual, second edition (Sambrook et al., 2000) Cold Spring Harbor Press; Current Protocols in Molecular Biology (F. M. Ausubel et al., eds., 1989) all of which are hereby incorporated by reference in their entirety.

As used herein, the singular form “a”, “an”, and “the” includes plural references unless indicated otherwise. For example, “a” symptom includes one or more or more symptoms.

A “subject” is a human subject.

As used herein, “treatment” is an approach for obtaining beneficial or desired clinical results. For purposes of this invention, beneficial or desired clinical results include, but are not limited to, one or more of the following: ameliorating one or more symptoms associated with acute myocardial infarction, cardioprotection, reduction in infarction size, reduction in reperfusion injury, one or more diagnostic indicators in acceptable clinical ranges, reduction in frequency of interventional therapies (e.g., PCI), delay in cardiovascular disease progression, such as but not limited to congestive heart failure, and/or improvement in quality of life.

“Prevention” refers to a reduction and/or delay in occurrence or reoccurrence of acute myocardial infarction in a subject at risk for an acute myocardial infarction compared. A subject at risk includes, but is not limited to, a subject with a family history of hypertension, cardiovascular disease or congestive heart failure or combinations thereof. By way of example, subjects in need of the treatment methods described herein for acute myocardial infarction may be administered a preventive maintenance therapy by the methods described herein.

“Ameliorating a symptom” includes a shortening or reduction in duration of a symptom, attenuation of a symptom, abolishment of the symptom or a delay in development or reoccurrence of the symptom. Symptoms of an AMI may include, but are not limited to, ischemic symptoms, such as for example, chest, epigastric, arm, wrist or jaw discomfort and/or pain; nausea; vomiting; weakness; dizziness; palpitations; cold perspiration; dyspnea; syncope and/or diaphoresis. (See, e.g., Braunwald, E et al (ed.) In Heart Disease—A Textbook of Cardiovascular Medicine, 6^{th} Edition, (2001) W.B. Saunders Company, Philadelphia; Chapter 35; Crawford, M H (ed.), In Current Diagnosis and Treatment of Cardiology, 2^{nd} Edition, (2003) Lange Medical Books/McGraw Hill, New York; Chapter 5).

“Cardioprotection” includes, but is not limited to, prevention, inhibition or reduction of myocardial cell necrosis resulting from an acute myocardial infarction and/or prevention, inhibition or reduction of myocardial cell damage.

“Diagnostic indicators” include, but are not limited to, rise and fall in biochemical markers indicative of myocardial cells becoming necrotic, such as for example, but not limited to, troponin and myocardial muscle creatinine kinase enzyme (CK-MB); development of pathologic Q waves on an electrocardiogram (ECG) and/or ST segment elevation or depression on an ECG (See, e.g., Braunwald, E et al (ed.) In Heart Disease—A Textbook of Cardiovascular Medicine, 6^{th} Edition, (2001) W.B. Saunders Company, Philadelphia; Chapter 35; Crawford, M H (ed.), In Current Diagnosis and Treatment of Cardiology, 2^{nd} Edition, (2003) Lange Medical Books/McGraw Hill, New York; Chapter 5).

An “effective amount” is generally an amount sufficient to effect beneficial or desired clinical results including, but not limited to, one or more of the following: ameliorating one or more symptoms associated with acute myocardial infarction; cardioprotection, reduction in infarction size, reduction in reperfusion injury, one or more diagnostic indicators in acceptable clinical ranges and/or improvement in quality of life.

As used herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline. Compositions comprising such carriers are formulated by well known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000; herein incorporated by reference in its entirety).

As used herein, administration “in conjunction” includes simultaneous administration and/or administration at different times. Administration in conjunction also encompasses administration as a co-formulation (e.g., CGRP and a second compound known to be useful for treating acute myocardial infarction) or administration as separate compositions. As used herein, administration in conjunction is meant to encompass any circumstance wherein CGRP and another compound, such as a compound known to be useful for the treatment of acute myocardial infarction, is administered to subject, which can occur simultaneously and/or separately. CGRP and any other compound can be administered at different dosing frequencies or intervals via the same route of administration or different routes of administration. Such compounds are suitably present in combination in amounts that are effective for the purpose intended.

### Methods of Treatment

This invention generally relates to a method of treating an acute myocardial infarction in a subject in need of such treatment. In one embodiment the method relates to treating a subject suspected of having an acute myocardial infarction comprising administering an effective amount of CGRP to a subject in need of such treatment. The criteria for diagnosing and evaluating subjects for acute myocardial infarction (AMI) are known in the art. Criteria for diagnosing and evaluating subjects for acute myocardial infarction may be found for example, in Braunwald, E et al (ed.) In Heart Disease—A Textbook of Cardiovascular Medicine, 6^{th} Edition, (2001) W.B. Saunders Company, Philadelphia; Chapter 35; Crawford, M H (ed.), In Current Diagnosis and Treatment of Cardiology, 2^{nd} Edition, (2003) Lange Medical Books/McGraw Hill, New York; Chapter 5; “Myocardial Infarction Redefined—A Consensus Document of The Joint European Society of Cardiology/American College of Cardiology Committee for redefinition of Myocardial Infarction” as published in the Journal of the American College of Cardiology, 36: 959-969 (2000); American Heart Association Guidelines for Acute Myocardial Infarction; and The American College of Cardiology Guidelines for Acute Myocardial Infarction, all incorporated by reference in their entirety herein). Exemplary non-limiting criteria are provided below.

Generally, by way of example and not limitation, a subject is suspected of having an AMI if the subject presents with one or more of the following symptoms: ischemic symptoms, such as by way of example, chest, epigastric, arm, wrist or jaw discomfort and/or pain; nausea; vomiting, weakness, dizziness, palpitations, cold perspiration, dyspnea, syncope, and/or diaphoresis. Diagnosis also generally involves assessment of various diagnostic indicators. Examples of diagnostic indicators include, but are not limited to rise and fall in biochemical markers indicative of myocardial necrosis, such as for example but not limited to, troponin and myocardial muscle creatinine kinase enzyme (CK-MB), development of pathologic Q waves on an electrocardiogram (ECG) and/or ST segment elevation or depression on an ECG. Generally, combinations of one or more symptoms and one or more diagnostic indicators are used in the evaluation of the patient. Generally, criteria for an established diagnosis of acute myocardial infarction include, but are not limited to, development of new pathologic Q waves on serial ECGs, normalization of biochemical markers of myocardial necrosis, and/or pathological findings of a healed or healing myocardial infarction.

The management of patients presenting with suspected acute myocardial infarction will generally vary depending on whether the patient's ECG shows an ST elevation or an ST, depression. (See for e.g., Ryan et al. AAC/AHA Guidelines for the Management of Patients with Acute Myocardial Infarction J. American College of Cardiology, September 1999). Generally, patients who have an ST elevation on ECG will be administered thrombolytics or sent for PCI if available at the facility where the patient has been admitted. Accordingly, in yet another embodiment the method relates to a method of treating an ST elevated acute myocardial infarction in a subject, comprising administering CGRP to a subject in need of such treatment.

Patients presenting with non-ST elevated ECG may require additional diagnostic indicators to confirm a diagnosis of AMI. Examples of such diagnostic indicators include, but are not limited to, rise and fall in biochemical markers indicative of myocardial necrosis and/or cardiac imaging. Cardiac imaging may be performed by methods known in the art, including, but not limited to, echocardiography and nuclear scanning. Accordingly, in yet another embodiment the method relates to a method of treating a non ST elevated acute myocardial infarction in a subject, comprising administering CGRP to a subject in need of such treatment.

For the methods of treatment, the CGRP administration can start at the initial stages of evaluation and treatment (e.g., paramedics, Emergency Room healthcare professional) of the subject. Administration can be by any means known in the art, including, for example, orally, intravenously, subcutaneously, intraarterially (such as via a coronary artery), intramuscularly, intracardially, intraspinally, intrathoracicly, intraperitoneally, intraventricularly, sublingually, via inhalation, injection and transdermally. In some embodiments, the CGRP is administered intravenously or via controlled release formulations.

By way of example, CGRP can be administered at a rate between about 0.8 ng/kg/min to about 16 ng/kg/min for up to about 24 hours or at a rate between about 4 ng/kg/min to about 10 ng/kg/min, such as for example, about 8 ng/kg/min for up to about 24 hours. In one embodiment the CGRP is administered between about 4 hours to about 12 hours or between about 6 hours to about 8 hours. By way of example, the CGRP can be administered at a rate of about 8 ng/kg/min for about 8 hours. Examples of routes of administration include, but are not limited to, intravenous administration and administration with controlled release formulations.

In another embodiment the CGRP is administered at a rate sufficient to achieve steady state plasma levels at between about 16 pg/ml to between about 314 pg/ml, for up to about 24 hours or between about 79 pg/ml to about 196 pg/ml, such as for example about 157 pg/ml, for up to about 24 hours. In one embodiment the CGRP is administered between about 4 hours to about 12 hours or between about 6 hours to about 8 hours. By way of example, the CGRP can be administered at a rate sufficient to achieve steady state plasma levels of about of about 157 pg/ml for about 8 hours. Examples of routes of administration include, but are not limited to intravenous administration and administration with controlled release formulations.

CGRP may be acting as a cardioprotective agent based on its ability to modulate cytokines during inflammation associated with AMI. Examples of cytokines inhibited or suppressed by CGRP, include, but are not limited to TNF-α, IL-1β, IL-7, IL-12, IL-16 and B7-2. Examples of anti-inflammatory cytokines induced by CGRP include, but are not limited to, IL-10. CGRP may be also be acting as a cardioprotective agent based on its ability to modulate cytokines during myocardial cell necrosis or cell damage during an AMI. Examples of such cytokines include, but are not limited to, IGF-1. Accordingly, in one embodiment, for cardioprotection in treating an acute myocardial infarction in a subject in need of such treatment, CGRP can be administered at a rate between about 0.8 ng/kg/min to about 16 ng/kg/min for up to about 24 hours or at a rate between about 4 ng/kg/min. to about 10 ng/kg/min, such as for example, about 8 ng/kg/min for up to about 24 hours. In one embodiment the CGRP is administered between about 4 hours to about 12 hours or between about 6 hours to about 8 hours. By way of example, the CGRP can be administered at a rate of about 8 ng/kg/min for about 8 hours. Examples of routes of administration include, but are not limited to intravenous administration and administration with controlled release formulations.

In another embodiment, for cardiprotection in treating an acute myocardial infarction in a subject in need of such treatment, CGRP is administered at a rate sufficient to achieve steady state plasma levels at between about 16 pg/ml to between about 314 pg/ml, for up to about 24 hours or between about 79 pg/ml to about 196 pg/ml, such as for example about 157 pg/ml, for up to about 24 hours. In one embodiment the CGRP is administered between about 4 hours to about 12 hours or between about 6 hours to about 8 hours. By way of example, the CGRP can be administered at a rate sufficient to achieve steady state plasma levels of about of about 157 pg/ml for about 8 hours. Examples of routes of administration include, but are not limited to intravenous administration and administration with controlled release formulations.

Efficacy of the treatment can be evaluated by medical personnel based on a variety of standard tests. Examples of such techniques include, but are not limited to, but are not limited to, measurement of biochemical markers indicative of myocardial necrosis, such as for example, troponin and myocardial muscle creatinine kinase enzyme (CK-NM), Q waves on an electrocardiogram (ECG); ST segment on an ECG; reduction in infarction size, and/or reduction in reperfusion injury. Amelioration of any one or more symptoms of AMI is indicative of the efficacy of the treatment.

In another embodiment CGRP is used in the manufacture of medicament for treating an acute myocardial infarction in a subject in need of such treatment or for treating a subject suspected of having a myocardial infarction in need of such treatment. The medicament may be administered by methods and dosages exemplified herein.

By way of example, the medicament can be administered to a subject at a rate between about 0.8 ng/kg/min to about 16 ng/kg/min for up to about 24 hours or at a rate between about 4 ng/kg/min to about 10 ng/kg/min, such as for example, about 8 ng/kg/min for up to about 24 hours. By way of example, the medicament may be administered between about 4 hours to about 12 hours or between about 6 hours to about 8 hours. Routes of administration include, but are not limited to, intravenous administration and administration with controlled release formulations.

Also by way of example, the medicament may be administered at a rate sufficient to achieve steady state plasma levels at between about 16 pg/ml to between about 314 pg/ml, for up to about 24 hours or between about 79 pg/ml to about 196 pg/ml, such as for example about 157 pg/ml, for up to about 24 hours. In one embodiment the CGRP is administered between about 4 hours to about 12 hours or between about 6 hours to about 8 hours. By way of example, the CGRP can be administered at a rate sufficient to achieve steady state plasma levels of about of about 157 pg/ml for about 8 hours. Examples of routes of administration include, but are not limited to intravenous administration and administration with controlled release formulations.

### Methods of Prevention

In one embodiment, the invention relates to a method of preventing an acute myocardial infarction in a subject in need of such treatment, comprising administering CGRP to a subject in need of such treatment at a rate between about 0.8 ng/kg/min to about 10 ng/kg/min continuously as needed. In another embodiment, the invention relates to a method of preventing an acute myocardial infarction, comprising administering CGRP to a subject in need of such treatment at a rate sufficient to achieve steady state plasma levels at between about 16 pg/ml to about 196 pg/ml continuously as needed.

A subject in need of preventive treatment includes a subject at risk of having an AMI or at risk of reoccurrence of an AMI. At risk subjects include, but are not limited to, a subject who has had one or more AMI, a subject with a family history of hypertension, cardiovascular disease, congestive heart failure or combinations thereof. By way of example, a subject in need of the treatment methods described herein for AMI may be administered as a preventive maintenance after the course of treatment for AMI as described herein. By way of example, a maintenance therapy for a subject in need of such treatment can comprise administration of CGRP at a rate between about 0.8 ng/kg/min to about 10 ng/kg/min continuously as needed. Also, by way of example, a maintenance therapy for a subject in need of such treatment can comprise administration of CGRP at a rate sufficient to achieve steady state plasma levels at between about 16 pg/ml to about 196 pg/ml continuously as needed.

CGRP may be acting as a cardioprotective agent based on its ability to modulate cytokines during inflammation associated with AMI. In one embodiment CGRP administration as maintenance therapy for cardioprotection in a subject in need of such treatment can comprise administration of CGRP at a rate between about 0.8 ng/kg/min to about 10 ng/kg/min continuously as needed. Also, by way of example, a maintenance therapy for cardioprotection to a subject in need of such treatment can comprise administration of CGRP at a rate sufficient to achieve steady state plasma levels at between about 16 pg/ml to about 196 pg/ml continuously as needed.

Administration can be by any means known in the art, including, for example, orally, intravenously, subcutaneously, intraarterially (such as via a coronary artery), intramuscularly, intracardially, intraspinally, intrathoracicly, intraperitoneally, intraventricularly, sublingually, via inhalation, injection and transdermally. In some embodiments, the CGRP is administered intravenously or via controlled release formulations. By way of example, intradermal administration, such as a depot or a controlled release formulation for continuous administration as needed may be used.

In another embodiment CGRP is used in the manufacture of medicament for preventing an acute myocardial infarction in a subject in need of such treatment. The medicament may be administered by methods and dosages exemplified herein.

By way of example, the medicament may be administered to the subject at a rate between about 0.8 ng/kg/min to about 10 ng/kg/min continuously as needed. Also, by way of example, the medicament may be administered at a rate sufficient to achieve steady state plasma levels at between about 16 pg/ml to about 196 pg/ml continuously as needed.

Administration of the medicament can be by any means known in the art, including, for example, orally, intravenously, subcutaneously, intraarterially (such as via a coronary artery), intramuscularly, intracardially, intraspinally, intrathoracicly, intraperitoneally, intraventricularly, sublingually, via inhalation, injection and transdermally. In some embodiments, the CGRP is administered intravenously or via controlled release formulations. By way of example, intradermal administration, such as a depot or a controlled release formulation for continuous administration as needed may be used.

### CGRP

The CGRP used in this invention may be synthetically or recombinantly produced or isolated from natural sources by methods known in the art. Preferably the alpha form of human CGRP (e.g., human α-CGRP or human CGRP-1; MW: about 3789 g/mol) is used in the methods described herein. An exemplary 37 amino acid sequence for human α-CGRP or human CGRP-1 is provided below:

Ala Cys Asp Thr Ala Thr Cys Val Thr His Arg Leu Ala
Gly Leu Leu Ser Arg Ser Gly Gly Val Val Lys Asn Asn
Phe Val Pro Thr Asn Val Gly Ser Lys Ala Phe

(see also for e.g., Morris et al., Nature 308 (5961), 746-748 (1984); GenBank Accesion No.: 1005250A).

Also contemplated by this invention are conservative substitutions to the CGRP. Conservative substitutions are known to those of skill in the art. Criteria for conservative substitutions include, but are not limited to, similar charge, polarity, hydrophobicity, stearic confirmation and bulkiness. Examples of substitutions that can be made to CGRP, such as to human α-CGRP, include, but are not limited those shown in Table 2.

TABLE 2

| Original Residue | Exemplary Substitutions |
|---|---|
| Ala | Val, Gly, Ser |
| Arg | Lys |
| Asn | Gln, His |
| Asp | Glu |
| Cys | Ser |
| Gln | Asn |
| Glu | Asp |
| Gly | Val, Ala, Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Synthetic CGRP, such as human α-CGRP, may be obtained using technology known in the art, for example, an automatic peptide synthesizer according to well known methods. One method for synthesizing the CGRP is the well known Merrifield method (see, Merrifield, R. B., J. Am. Chem. Soc., 85:2149 (1963) and Merrifield, R. B., Science, 232:341 (1986), which are specifically incorporated herein by reference). Recombinantly produced human α-CGRP may also be produced by methods known in the art. (e.g., see Molecular Cloning: A Laboratory Manual, second edition (Sambrook et al., 2000) Cold Spring Harbor Press; Current Protocols in Molecular Biology (F. M. Ausubel et al., eds., 1989)

Alternatively, human CGRP also may be obtained from commercial sources, such as Peninsula Laboratory (Belmont, Calif.), Bachem Biosciences, Inc. (King of Prussia, Pa.) and Sigma Chemicals (St. Louis, Mo.). Commercial grade human CGRP can require purification and sterilization so that it is fit for human use.

CGRP analogs are also contemplated in this invention. By way of example, and not limitation, a CGRP analog based on the CGRP receptor structure can be used. Examples of CGRP analogs, include, but are not limited to, peptide-based analogues, and peptide-mimetic analogs. Analogs of CGRP preferably retain the activity of CGRP, such activity may be evaluated by measuring cAMP levels in cell culture models as known in the art. An example of such as assay may be found in Nishikimi T et al, Effect of adrenomedullin on cAMP and cGMP levels in rat cardiac myocytes and nonmyocytes. Eur J. Pharmacol. 1998 Jul. 24; 353(2-3):337-44, herein incorporated by reference in its entirety.

CGRP may be acting as a cardioprotective agent based on its ability to modulate cytokines during inflammation associated with AMI. Examples of cytokines inhibited or suppressed by CGRP (see Table 1 below) include TNF-α, IL-1β, IL-7, IL-12, IL-16 and B7-2 (Feng et al, (1997) Life Sci. 61(20):PL281-7; Torii H et al (February 1997) Leukoc Biol 61(2):216-23; Fernandez S et al (2000) Leukoc Biol 67(5):669-76; Dunzendorfer S et al (2002-2003) Neuroimmunomodulation 10(4):217-23; Ashana et al (August 1995) PNAS (USA) 92:8323-8327, herein incorporated by reference in their entirety.) IL-10 is an anti-inflammatory cytokine induced by CGRP (Torii H et al (February 1997) Leukoc Biol 61(2):216-23, herein incorporated by reference in its entirety). Accordingly an analog of CGRP may be identified by its ability to inhibit or suppress TNF-α, IL-1β, IL-7, IL-12, IL-16 and B7-2 or its ability to induce IL-10 in assays. Examples of assays to measure induction or suppression of cytokines are known in the art. By way of example, the assays described in the references in Table 1 may be used to evaluate the analog.

TABLE 1

| CGRP | |
|---|---|
| ACTIVITY | Benefits |
| Cytokines | |
| inhibited or | |
| suppressed | |
| TNF-α | Inhibits lipopolysaccharide induced TNF-α in macrophages |
| | Feng et al, (1997) Life Sci. 61(20): PL281-7. |
| IL-1β | Inhibits proliferation of T-cells |
| | Torii H et al (February 1997) Leukoc Biol 61(2): 216-23 |
| IL-7 | Inhibits pre B-cell development and T-cell development |
| | Fernandez S et al (2000) Leukoc Biol 67(5): 669-76 |
| IL-12 | Suppresses Natural Killer Cell Stimulating factor (NKSF) |
| | Torii H et al (February 1997) Leukoc Biol 61(2): 216-23 |
| IL-16 | Inhibits macrophage chemotaxis |
| | Dunzendorfer S et al (2002-2003) |
| | Neuroimmunomodulation 10(4): 217-23 |
| B7-2 | Inhibits Granulocyte-Macrophage Colony |
| | Stimulating Factor (GM-CSF) expression |
| | Ashana et al (August 1995) PNAS (USA) 92: 8323-8327 |
| | Torii H et al (February 1997) Leukoc Biol 61(2): 216-23 |
| Induced | |
| anti-inflam- | |
| matory | |
| cytokines | |
| IL-10 | Inhibits monocyte inflammation |
| | Torii H et al (February 1997) Leukoc Biol 61(2): 216-23 |

CGRP may be also be acting as a cardioprotective agent based on its ability to modulate cytokines during myocardial cell necrosis or cell damage during an AMI. Examples of such cytokines include, but are not limited to, IGF-1. CGRP increases expression of IGF-1 (insulin-like growth factor-1), which in turn upregulates extracellular receptor kinase 1 and 2 (ERK 1/2) which appears to protect cardiac myocytes and vascular smooth muscle from oxidative stress induced apoptosis (R. Zfoncea et al (August 1997) J. Biol. Chem. 273(31):19115-19124). Examples of assays to measure induction of cytokines are known in the art. Examples of assays for evaluating cell necrosis or cell damage are also known in the art

Other forms of CGRP which are suitable for use in the methods of this invention include pharmaceutically acceptable prodrugs of CGRP. A “pharmaceutically acceptable prodrug” is a compound that may be converted under physiological conditions or by solvolysis to the specified compound or to a pharmaceutically acceptable salt of such compound. Prodrugs of CGRP may be identified using routine techniques known in the art. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the present invention. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews 1996, 19, 115. Carbamate prodrugs of hydroxy and amino groups are also included, as are carbonate prodrugs, sulfonate esters and sulfate esters of hydroxy groups. Derivitization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities. Other examples of such prodrug derivatives are described in a) Design of Prodrugs, edited by H. Bundgaard, (Elsevier, 1985) and Methods in Enzymology, Vol. 42, p. 309-396, edited by K. Widder, et al. (Academic Press, 1985); b) A Textbook of Drug Design and Development, edited by Krogsgaard-Larsen and H. Bundgaard, Chapter 5 “Design and Application of Prodrugs”, by H. Bundgaard p. 113-191 (1991); c) H. Bundgaard, Advanced Drug Delivery Reviews, 8:1-38 (1992); d) H. Bundgaard, et al., J. Pharmaceutical Sciences, 77:285 (1988); and e) N. Kakeya, et al., Chem. Pharm. Bull., 32:692 (1984), each of which is specifically incorporated herein by reference.

The CGRP peptide of the invention can also be conjugated with one or more chemical groups. The chemical groups utilized for conjugation are preferably not significantly toxic or immunogenic. Exemplary chemical groups include carbohydrates, such as, for example, those carbohydrates that occur naturally on glycoproteins, and non-proteinaceous polymers, such as polyols.

A polyol, for example, can be conjugated to the peptide at one or more amino acid residues. The polyol employed can be any water-soluble poly(alkylene oxide) polymer and can have a linear or branched chain. Examples of suitable polyols include, but is not limited to, a poly(alkylene glycol), such as poly(ethylene glycol) or PEG. The process of conjugating the polyol to a peptide is termed “pegylation.” Those skilled in the art recognize that other polyols, such as, for example, poly(propylene glycol) and polyethylene-polypropylene glycol copolymers, can be employed using the techniques for conjugation described herein for PEG. A variety of methods for pegylating proteins have been described. See, e.g., U.S. Pat. No. 4,179,337. Suitable PEGS for use in the methods described herein may be made by conventional methods or alternatively, purchased commercially. The degree of pegylation of the invention can be adjusted to provide a desirably increased in vivo half-life, compared to the corresponding non-pegylated protein. It is believed that the half-life of a pegylated CGRP typically increases incrementally with increasing degree of pegylation.

If the CGRP is synthetically made, the terminal caboxy and amino groups may comprise any of the end groups generated during protein synthesis (see, e.g., Lloyd-Williams et al (eds.) (1997) Chemical Approaches to the Synthesis of Peptides and Proteins; CRC Press)

### Pharmaceutical Compositions

The CGRP composition used in the present invention can further comprise pharmaceutically acceptable carriers, excipients, or stabilizers (Remington: The Science and practice of Pharmacy 20th Ed. (2000) Lippincott Williams and Wilkins, Ed. K. E. Hoover), in the form of lyophilized formulations or aqueous solutions. Acceptable carriers, excipients, or stabilizers are nontoxic to recipients at the dosages and concentrations, and may comprise buffers such as phosphate, citrate, acetate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium or acetate; metal complexes (e.g. Zn-protein complexes); and/or non-ionic surfactants such as TWEEN™, PLURONICS™ or polyethylene glycol (PEG).

### Intravenous

CGRP or a pharmaceutically acceptable formulation thereof may be formulated for parenteral administration, e.g., for intravenous, subcutaneous, or intramuscular injection. By way of example, an intravenous formulation may be used in the methods described herein comprises saline and about 0.05% polysorbate AB (e.g., TWEEN-80). For perenteral administration, such as intravenous administration, a dose of CGRP may be combined with a sterile aqueous solution which is preferably isotonic with the blood of the patient. Such a formulation may be prepared by dissolving a solid active ingredient in water containing physiologically-compatible substances such as sodium chloride, glycine, and the like, and having a buffered pH compatible with physiological conditions so as to produce an aqueous solution, and then rendering the solution sterile by methods known in the art. The formulations may be present in unit or multi-dose containers, such as sealed ampules or vials. The formulation may be delivered by any mode of injection, including, without limitation, epifascial, intracutaneous, intramuscular, intravascular, intravenous, parenchymatous, subcutaneous, oral or nasal preparations (see, for example, U.S. Pat. No. 5,958,877, which is specifically incorporated herein by reference).

In one embodiment the formulation for parenteral administration is designed to achieve steady state plasma levels at between about 16 pg/ml to between about 314 pg/ml, for up to about 24 hours or between about 79 pg/ml to about 196 pg/ml, such as for example about 157 pg/ml, for up to about 24 hours when administered. In another embodiment, the formulation for parenteral administration is designed to administer between about 0.8 ng/kg/min to about 16 ng/kg/min for up to about 24 hours or at between about 4 ng/kg/min to about 10 ng/kg/min, such as for example, about 8 ng/kg/min for up to about 24 hours.

### Controlled Release

In another embodiment, this invention relates to controlled release formulations of CGRP. By way of example and not limitation, the CGRP controlled release formulations may comprise controlled release formulations of polymers as set forth in U.S. Pat. Nos. 5,702,716 (Dunn et al); 5,324,519 (Dunn et al) or 6,143,314 (Chandrashekar). In one embodiment the formulation for controlled release is designed to achieve steady state plasma levels at between about 15.7 pg/ml to between about 314 pg/ml, for up to about 24 hours or between about 78.5 pg/ml to about 196 pg/ml, such as for example about 157 pg/ml, for up to about 24 hours when administered.

### Combination Therapies

The CGRP compositions of the invention can also be administered in conjunction with other compounds known to be useful for the treatment of AMI. CGRP can serve to enhance and/or complement the effectiveness of such compounds. Accordingly, the compositions described herein may be administered in conjunction with one or more additional compounds known to be useful for the treatment of AMI, including but not limited to: beta-blockers, antithrombolytic agents, angiotensin converting enzyme (ACE) inhibitors, calcium channel blockers, nitrates, aspirin, opioids (e.g., morphine), and non-steroidal anti-inflammatories. Such compounds are suitably present in amounts that are effective for the purpose intended.

Examples of beta blockers include, but are not limited to, 2-[p-[2-hydroxy-3-(isopropylamino)propoxy]phenyl]acetamide (e.g., Atenolol), (S)-1-[(1,1-dimethylethyl)amino]-3-[[4-(4-morpholinyl)-1,2,5-thiadiazol-3-yl]oxy]-2-propanol, (Z)-2-butenedioate (1:1) salt (e.g., Timolol), 1-[(1-methylethyl)amino]-3-[2-(2-propenyloxy)phenoxy]-2-Propanol, (e.g., Alprenolol), 1-(isopropylamino)-3-(1-naphthyloxy)-2-propanol (e.g., Propranolol), 1-(isopropylamino)-3-[p-(2-methoxyethyl)phenoxy]-2-propanol tartrate (e.g., Metoprolol), and methyl-4-[2-hydroxy-3-[(1-methylethyl)amino]-propoxy]benzenepropanoate (e.g., Esmolol).

Examples of antithrombolytic agents include, but are not limited to, 2-acetoxybenzoic acid (e.g., Aspirin), 5-(o-chlorobenzyl)-4,5,6,7-tetrahydrothieno-[3,2-c]pyridine (e.g., Ticlopidine), methyl (+)-(S)-a-(o-chlorophenyl)-6,7-dihydrothieno[3,2-c]pyridine-5(4H)-acetate (e.g., Clopidogrel), heparin (unfractionated heparin and low molecular weight heparins, such as nadroparin, dalteparin (fragmin), enoxaparin), streptokinase, anistreplase, alteplase, reteplase, tissue plasminogen activator (t-PA), TNK-tissue plasminogen activator (TNK-tPA), lanoteplase, abciximab, and hirudin. Examples of angiotensin converting enzyme (ACE) inhibitors include, but are not limited to, 1-[(2S)-3-mercapto-2-methylpropionyl]-L-proline (e.g., Captopril), (2S,3aS,6aS)-1-[(S)—N-[(S)-1-carboxy-3-phenylpropyl]alanyl]octahydrocyclopenta[b]-pyrrole-2-carboxylic acid (e.g., Ramipril), N-[(S)-1-carboxy)-3-phenylpropyl]-L-alanyl-L-proline (e.g., Zofenopril), 1-[N-[(S)-1-carboxy-3-phenylpropyl]-L-alanyl]-L-proline 1′-ethyl ester (e.g., Enalapril), and (S)-2-[(S)-N-[(S)-1-Carboxy-3-phenylpropyl]alanyl]-1,2,3,4-tetrahydro-3-isoquinolinecarboxylic acid 1-ethyl ester (e.g., Quinapril).

Examples of calcium channel blockers include, but are not limited to, 5-[(3,4-dimethoxyphenethyl)methylamino]-2-(3,4-dimethoxyphenyl)-2-isopropylvaleronitrile (e.g., Verapamil) and (+)-5-[2-(dimethylamino)ethyl]-cis-2,3-dihydro-3-hydroxy-2-(p-methoxyphenyl)-1,5-benzothiazepin-4(5H)-one acetate (ester) (e.g., Diltiazem). Examples of nitrates include, but are not limited to, 1,2,3-propanetriol trinitrate (e.g., Nitroglycerin), isosorbide dinitrate (ISDN), and isosorbide-5-mononitrate (ISMN).

### Kits

The invention also provides kits for use in the instant methods. Kits of the invention include one or more containers comprising CGRP and instructions for use in accordance with any of the methods described herein and preferably a delivery device (e.g., minipump or other controlled release formulation) for the CGRP. The instructions may also comprise a description of selecting a subject for treatment based on identifying whether that subject is, for example, suspected of having an AMI or of having had an AMI. In some embodiments, the instructions comprise description of administering CGRP to the subject in need of treatment for an AMI or suspected of having an AMI.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump or other controlled release formulation. A kit may have a sterile access port (for example the container may be an intravenous solution bag or a vial having a stopper pierceable by a hypodermic injection needle).

The instructions relating to the use CGRP generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

In some embodiments, the kit comprises a container and a label or package insert(s) on or associated with the container. The container holds a CGRP composition which is effective for any of the methods described herein. By way of example, one or more of the container may comprise lyophilized CGRP and one or more containers may comprise a suitable carrier for resuspending the CGRP. Also by way of example, and not limitation, one or more containers can comprise CGRP in solution form or in controlled release formulation. The container may further comprise a second pharmaceutically active agent known to be useful in the treatment of AMI. Kits may optionally provide additional components such as buffers and interpretive information.

Although the foregoing invention has been described in some detail by way of illustration and example for purposes of clarity of understanding, it will be apparent to those skilled in the art that certain changes and modifications may be practiced. Therefore, the descriptions and examples should not be construed as limiting the scope of the invention.

1-21. (canceled)

22: A method for treating an acute myocardial infarction in a subject, comprising administering CGRP to a subject in need of such treatment.

23: The method of claim 22, wherein the CGRP is administered to the subject at rate between about 0.8 ng/kg/min to about 16 ng/kg/min for up to about 24 hours.

24: The method of claim 22, wherein the CGRP is administered to the subject at a rate between about 4 ng/kg/min to about 10 ng/kg/min for up to about 24 hours.

25: The method of claim 22, wherein the CGRP is administered to the subject at a rate of about 8 ng/kg/min to about 10 ng/kg/min for up to about 24 hours.

26: The method of claim 22, wherein the CGRP is administered to the subject at a rate sufficient to achieve steady state plasma levels at between about 16 pg/ml to between about 314 pg/ml for up to 24 hours.

27: The method of claim 22, wherein the CGRP is administered to the subject at a rate sufficient to achieve steady state plasma levels at between about 79 pg/ml to between about 196 pg/ml for up to 24 hours.

28: The method of claim 22, wherein the CGRP is administered to the subject at a rate sufficient to achieve steady state plasma levels of about 196 pg/ml for up to 24 hours.

29: A method for treating a non-ST elevated acute myocardial infarction, comprising administering CGRP to a subject in need of such treatment.

30: A method for treating a ST elevated acute myocardial infarction, comprising administering CGRP to a subject in need of such treatment.

31: A method for treating a subject suspected of having an acute myocardial infarction, comprising administering CGRP to a subject in need of such treatment.

32: The method of claim 31, wherein the CGRP is administered to the subject at rate between about 0.8 ng/kg/min to about 16 ng/kg/min for up to about 24 hours.

33: The method of claim 31, wherein the CGRP is administered to the subject at a rate between about 4 ng/kg/min to about 10 ng/kg/min for up to about 24 hours.

34: The method of claim 31, wherein the CGRP is administered to the subject at a rate of about 8 ng/kg/min to about 10 ng/kg/min for up to about 24 hours.

35: The use of claim 31, wherein the CGRP is administered to the subject at a rate sufficient to achieve steady state plasma levels at between about 16 pg/ml to between about 314 pg/ml for up to 24 hours.

36: The method of claim 31, wherein the CGRP is administered to the subject at a rate sufficient to achieve steady state plasma levels at between about 79 pg/ml to between about 196 pg/ml for up to 24 hours.

37: The method of claim 31, wherein the CGRP is administered to the subject at a rate sufficient to achieve steady state plasma levels of about 157 pg/ml for up to 24 hours.

38: A method for preventing an acute myocardial infarction in a subject, comprising administering CGRP to a subject in need of such treatment.

39: The method of claim 38, wherein the CGRP is administered to the subject at rate between about 0.8 ng/kg/min to about 10 ng/kg/min continuously as needed.

40: The method of claim 38, wherein the CGRP is administered to the subject at a rate sufficient to achieve steady state plasma levels of about 16 pg/ml to about 196 pg/ml continuously as needed.

41: The method of claim 22, wherein
the CGRP is administered as a controlled release formulation.

42. The method of claim 22, wherein the CGRP is administered as an intravenous formulation.

43. (canceled)


SEQUENCE LISTING (1 sequence)

SEQ ID NO: 1 - protein, 37 aa, Artificial Sequence
  Synthetic Construct
ACDTATCVTHRLAGLLSRSGGVVKNNFVPTNVGSKAF